(19) United States
(12) Patent Application Publication
(10) Pub. No.: US 2025/0235417 A1
(43) Pub. Date: Jul. 24, 2025

(54) Compositions for acute and chronic wounds

(71) Applicant: DEBx Medical Holding B.V., Amsterdam (NL)

(72) Inventors: Carlo Alberto Bignozzi, Ferrara (IT); Alberto Cogo, Vicenza (IT); Bertus Jozef Quint, Amsterdam (NL)

(73) Assignee: DEBx Medical Holding B.V., Amsterdam (NL)

(21) Appl. No.: 18/698,033

(22) PCT Filed: Jun. 27, 2022
(86) PCT No.: PCT/EP2022/067593

§ 371 Date: Apr. 3, 2024

(30) Foreign Application Priority Data
Oct. 7, 2021 (IT) 102021000025541

(51) Int. Cl. A61K 31/185; A61K 9/00; A61K 9/06; A61P 17/02

(52) U.S. Cl. CPC A61K 31/185; A61K 9/0014; A61K 9/06; A61P 17/02

(57) ABSTRACT

The invention concerns a sulphonic acid having the general formula (A):

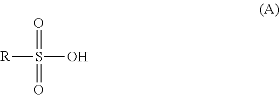

wherein R is an arene group, preferably a benzene group, wherein the arene group or benzene group is optionally substituted with one or more substituents independently selected from alkyl, OH and alkoxy, such as methoxy, wherein the substituent is preferably alkyl having a number of carbon atoms from 1 to 20 and the alkyl chain is at position 3 or 4 of the benzene ring, for use in removing a bacterial biofilm and/or necrotic or infected tissue from acute or chronic, preferably chronic, skin lesions or from oral cavities, or for use in disinfecting acute or chronic skin lesions.

The invention also concerns a composition comprising the sulphonic acid having the general formula (A) and at least one proton acceptor selected from: dimethyl sulfoxide, isopropyl alcohol, 3-methoxy-3-methyl-1-butanol, propylene carbonate, anhydrous sodium carbonate, ethylenediaminetetraacetic acid tetrasodium salt, sodium gluconate, polyethylene glycol, anhydrous silicon dioxide, tetraethoxysilane, and mixtures thereof.

### FIELD OF THE INVENTION

The invention concerns composition usable by medical staff (doctors/nurses) or also directly usable by the patient themselves (or their caretakers) for the cleansing and/or disinfection of skin wounds, both acute and chronic, and for the removal of the biofilm present in superficial bodily lesions, in particular wounds and chronic ulcers, or for removal necrotic or infected tissues from the oral cavity, in particular from periodontal pockets, peri-implant pockets or from wounds.

### BACKGROUND ART

It is well-known that skin wounds, both acute and chronic, contain microorganisms, in particular bacteria, that replicate and cause a persistent inflammatory response. In a chronic lesion, the continuous presence of viral microorganisms leads to a heavy and persistent inflammatory response, which in the end contributes to damage the host organism. Indeed, a persistent production of inflammation mediators and a constant migration of neutrophile granulocytes occur. The latter release in the wound cytolytic enzymes and free radicals, which are the main cause of tissue damage. Furthermore, macrophages are inhibited with a consequent inability of the chronic lesion to auto regenerate. Moreover, a localised thrombosis may occur and metabolites with vasoconstrictor action are released which can induce tissue hypoxia, causing further bacterial proliferation and tissue damage. Due to the considerable invasiveness of some infective bacterial species, the microbic component may contribute to aggravate the lesions and increase the layer of biofilm.

The term “biofilm” defines the thin layer of polysaccharides, nucleic acids, proteins, lipids and glycoprotein material that is produced by bacteria in active, albeit slow, replication and that adheres to the lesion bed. The biofilm that is formed in an infected wound contributes to delaying its healing. Indeed, in the presence of biofilm, the conditions are created in order for the individual microorganisms to interact reciprocally exchanging nutrients and metabolites, so that to establish actual organized bacterial communities. Therefore, biofilms act as protected infection and bacterial resistance outbreaks within the wound and they are able to protect bacteria from the action of antimicrobial agents, such as antibiotics and antiseptics. Biofilms usually consist of a plurality of layers of microorganisms and communication between microbial cells is a crucial factor for the development and maintenance of the biofilm itself.

The term “chronic skin ulcer” defines the skin lesions that do not progress towards healing after at least six weeks of appropriate treatment. Unrelated to aetiology (there are indeed chronic ulcers that are diabetic, venous, arteriopathic, vasculitic and post-traumatic), chronic skin ulcers show some common pathogenic mechanisms, which make them chronic. Among these pathogenic mechanisms, the most well-known are: the excessive presence of inflammatory proteins (cytokines and proteases) in the exudate; a persistent colonization of the bottom of the lesion by pathogenic microorganisms; the development of a biofilm which makes the bottom of the lesion inaccessible to drugs and dressings. These characteristics of chronic skin ulcer slow down or stop the autolytic process of remotion of the non-viable material, the proliferation of new vessels, which is essential for the formation of the granulation tissue, and the proliferation of dermal and epidermal cells.

Chronic skin ulcers, for example chronic skin ulcers of the inferior limbs, are a worldwide-spread problem. It is indeed estimated that 1.5% of the world population is affected by such pathology. Since age is a significant risk factor, the prevalence of said chronic skin ulcers is low in paediatric age and very high in elderly age.

In addition to what has been highlighted above with specific reference to the problem of the chronic cutaneous ulcers, it should be observed that the phenomena of microbial proliferation are further responsible for, or involved in, pathologies relating to the oral cavity, such as the periodontal disease and the perimplantitis.

The term “periodontal disease” defines a group of inflammatory pathologies that attack the system of tooth supporting tissues or periodontium, which can be distinguished in superficial periodontium (gingiva) and deep periodontium (periodontal ligament, root cementum and alveolar bone). The most frequent cause of periodontal disease is microbial, in particular bacterial, and the microorganisms involved are those normally present in the bacterial plaque. The infection occurs when—due to an excessive bacterial proliferation and/or a reduction of the organism's defensive mechanisms—the normal equilibrium is lost that keeps the tissues healthy. When the inflammation involves only the superficial periodontium the clinical picture is gingivitis, when on the other hand (in the absence of a suitable treatment) the inflammation extends beyond the gingival region, affecting the deep zones (alveolar bone, periodontal ligament and cement) of the periodontium, the clinical picture is periodontitis. Gingivitis attacks the gingiva near the tooth (marginal gingiva) and the symptoms (which are completely reversible after suitable therapeutic treatment) comprise reddening of the gingival margin, oedema and bleeding following mechanical stimulation. In the periodontitises the tooth supporting system is destroyed and it is manifested by attachment and bone loss, formation of pockets and shrinkage of the gingiva. The typical sign of the periodontitises is the formation of a periodontal pocket associated with dental looseness. The destruction of the teeth supporting tissues is in most cases irreversible. The periodontitis is always preceded by gingivitis and so by preventing gingivitis it is possible to prevent the periodontitis.

Gingivitis and periodontitis are pathologies that show an essentially bacterial but multifactorial aetiology, in which three cofactors interact: susceptibility of the host, environmental factors and behavioural factors. The bacterial plaque, although being necessary to the onset of the periodontal disease, is influenced by the interaction with the host and by numerous local and systemic factors, such as for example diabetes, which affect the clinical course. In Italy, the periodontal disease affects about 60% of the population and the individuals between 35 and 44 years of age are particularly affected. This percentage includes both the surface form (gingivitis, which affects the portion of gingiva near the tooth or marginal gingiva) and the deep form (periodontitis proper).

The treatment of the periodontal disease has to aim to stop the progression of the disease and prevent or reduce the onset of possible recurrences. In particular, the periodontitis therapy has to take into account the multifactorial nature of the pathology and provide clinical treatments that are able to face the complexity of the problem. The control of the causal agents is pursued and achieved through the mechanical removal (for example, by curettes or ultrasound devices) of the supragingival and subgingival bacterial plaque, as well as through the possible use of topical or systemic drugs. Among the periodontal pathogenic microorganisms the following species are counted: Aggregatibacter actinomycetemcomitans, Porphyromonas gingivalis, Tannerella forsythia and Treponema denticola.

From what has been highlighted above, it is clear that in order to oppose effectively the periodontal disease, it is necessary to reduce the bacterial load in the oral cavity.

The term “perimplantitis” defines an inflammatory process that attacks the tissues adjacent to an osscointegrated implant (artificial root that acts as a support for a fixed dental prosthesis) and leads to a destruction and loss of alveolar bone tissue. The causes are variable and may relate both to the surgical procedure of implanting the prosthesis and to the accompanying bacterial infections. It should nevertheless be noted that the bacterial aetiological component aggravates the problems connected to the surgical implant of the prosthesis. In order to safeguard the osseointegration process, the administration of antibiotic and anti-inflammatory drugs, a possible surgical treatment (so-called curettage) of the concerned area, as well as the guided bone regeneration, are used.

All the treatments aim to eliminate the bacteria from the surface of the implant, both blind technique (i.e. without piercing the visible gingiva) and open surgery (i.e. piercing and raising the gingiva). The blind technique treatments are less invasive but have cannot be associated with the bone regeneration, which always requires lifting of a gingiva flap. Furthermore, they are treatments that are conducted in a substantially “blind” manner, which do not enable to view and check whether the microbial load in the treated areas has actually been minimized.

The treatments of mechanical type provide for the use of curettes, sodium bicarbonate jet devices or powder jet devices, milling machine (for removing all the surface coils and roughnesses of the implant). The non-mechanical treatments provide for the use of antiseptic and antibiotic solutions, both as mouthwashes and as compositions that release active principles slowly. The laser can be used to sterilize the surfaces physically.

A drawback of all the treatments that limit themselves to eliminating the bacteria by disinfecting chemically or sterilizing physically is that the bacteria will again colonize the disinfected or sterilized zones. This is because the bacteria are not only on the implants but are substantially ubiquitous in the oral cavity.

It should also be noted that, both in the perimplantitis and in the periodontal disease of medium to severe degree, it is often necessary to accomplish a surgical therapy (debridement under local anaesthesia) to remove the infected tissue adjacent to the implant or to the tooth. Nevertheless, the surgical procedure alone is not able to remove the biofilm completely that forms in the lesion, so that it is necessary to associate the administration of antibiotics and disinfectants with the debridement.

In the context of the pathologies of the oral cavity aphthae have also to be mentioned, because of the particular frequency thereof. The term “aphtha” defines a painful ulcer caused by a breakage of the mucosa inside the oral cavity. These ulcers form periodically and can heal completely between one episode and the next. In most cases, the individual aphthae have a duration varying between 8 and 10 days. Most of aphthae appear on the surfaces of the non-keratinized epithelia of the mouth and, more specifically, everywhere except for adherent gingiva, hard palate and back of the tongue. The symptoms of the aphthae vary from a mild discomfort up to difficulty in eating and drinking. The condition is very common and affects about 20% of the population. The formation of aphthae has often infantile or adolescent onset and the condition usually lasts several years before gradually disappearing. Currently no definitive treatment has yet been identified for the aphtha and the treatment focuses on pain management, on the reduction of the healing times and on the reduction of the frequency of recurrences.

From what has been disclosed above, it is clear that the optimum treatment of skin pathologies (chronic cutaneous ulcers) and pathologies of the oral cavity (periodontal disease, perimplantitis, aphtha) requires a drastic reduction of bacterial contamination, which in turn requires an effective removal of the biofilm and of the necrotic or infected tissues. Nevertheless, the most used surgical method for removing necrotic or infected tissues, i.e. the debridement, has numerous drawbacks (risk of bleeding for the patient, need for hospitalization, complexity and high costs of execution).

In recent years, a considerable number of studies has highlighted the contribute of lipids to the microbic pathogenicity and to the formation of biofilm. These molecules constitute an important constituent of extracellular tissue that protects the bacterial and fungal biofilm from aggression by its potentially lytic agents. The dense extracellular matrix which encloses the biofilm is mainly composed of glycoproteins (55%), carbohydrates (25%), nucleic acids (5%) and lipids (15%), mainly glycerolipids and sphingolipids, which are crucial to cell adhesion to surfaces and to the formation of biofilm. Given the aforementioned composition, the use of anionic surfactants, which are components of soaps and detergents, should allow the solvation of the components of the biofilm and its consequent decomposition. However, tests performed with different types of soaps applied to chronic wounds have never given the desired results. Instead, previous studies have revealed the extraordinary activity of sulphonic acids like methanesulphonic acid, ethanesulphonic acid and propanesulphonic acid (PCT/IB2019/051146, PCT/EP2020/051652) in destroying the biofilm present in chronic wounds and also in periodontal pockets. It has been observed that the destruction of the biofilm is then followed by the spontaneous progressive healing of the treated wound.

Nonetheless, a desire exists for providing alternative compositions which can be used for destruction and removal in an equally efficient manner, of the biofilm and necrotic or infected tissues present in skin ulcers, while denaturing the microbial species present.

In a particular example, a presence of an exothermic effect upon contact with the biofilm composed of 97% of water, due to the dissociation of protons and the corresponding sulphonic anion, together with a burning sensation depending on the contact time, requires the application of these preparations by specialized medical-nursing staff.

Therefore, there is an additional need for new compositions that allow to remove in a less aggressive manner, and hence possibly autonomously by the patient, but in an equally efficient manner, the biofilm and necrotic or infected tissues present in skin ulcers, while denaturing the microbial species present.

### SUMMARY OF THE INVENTION

The invention concerns the use of a composition comprising a sulphonic acid having the general formula (A):

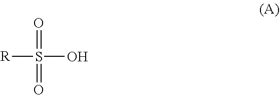

wherein R is an arene group, preferably a benzene group, wherein the arene group or benzene group is optionally substituted with one or more substituents independently selected from alkyl, OH and alkoxy, such as methoxy, wherein the substituent is preferably alkyl having a number of carbon atoms from 1 to 20 and the alkyl chain is at position 3 or 4 of the benzene ring, for use in removing a bacterial biofilm and/or necrotic or infected tissue from acute or chronic, preferably chronic, skin lesions or from oral cavities.

In another aspect the invention concerns the use of a composition comprising a sulphonic acid having the general formula (A):

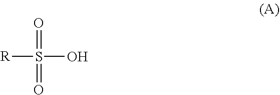

wherein R is an arene group, preferably a benzene group, wherein the arene group or benzene group is optionally substituted with one or more substituents independently selected from alkyl, OH and alkoxy, such as methoxy, wherein the substituent is preferably alkyl having a number of carbon atoms from 1 to 20 and the alkyl chain is at position 3 or 4 of the benzene ring, for use in disinfecting acute or chronic skin lesions.

The aryl sulphonic acids are in general strong with acidities however which are dependent by the substituents. Exemplary sulphonic acids according to the invention and their pKₐ and acid dissociation constant Kₐ are given in Table 1.

Preferably, the composition further comprises a non-aqueous proton acceptor. The inventors have found that combining a sulphonic acid with a non-aqueous proton acceptor provides a surprising combined effect of removing bacterial biofilm and necrotic and/or infected tissues present in skin lesions while preventing any damage to the skin lesion and the surrounding skin caused by the acidic activity of the sulphonic acid.

A composition containing the sulphonic acid and a non-aqueous proton acceptor has proven particularly useful in removing bacterial biofilm and necrotic and/or infected tissues present in skin lesions, preferably in acute or chronic skin ulcers.

Especially in case of a strong sulphonic acid the strong acidic action, which may provide aggressive action, is desirably compensated in the composition by adding the non-aqueous proton acceptor.

It has furthermore been found by the inventors, that when adding water to the composition, instead of the non-aqueous proton acceptor, considerably obstructs the composition of being effective in removing the biofilm and necrotic and/or infected tissues present in skin lesions.

Antimicrobial effects have been reported for several sulphonic acid compounds and compositions thereof. However, given that these known compositions contain water in a considerable amount (at least 10 wt. % or more) and they do not contain a non-aqueous proton acceptor, these compositions are not effective towards removing the biofilm and necrotic and/or infected tissues present in skin lesions. In those cases additional mechanical, i.e. surgical, debriment actions have been found to be needed to remove the biofilms.

In preferred embodiments, the non-aqueous proton acceptor is at least one component, selected from: dimethyl sulfoxide, isopropyl alcohol, 3-methoxy-3-methyl-1-butanol, propylene carbonate, anhydrous sodium carbonate, ethylenediaminetetraacetic acid tetrasodium salt, sodium gluconate, anhydrous silicon dioxide, tetraethoxysilane, polyethylene glycol and mixtures thereof. Preferably, the non-aqueous proton acceptor is dimethyl sulfoxide.

In another aspect, the invention concerns the use of a weak sulphonic acid, preferably an alkylbenzenesulphonic acid, preferably having an acid dissociation constant, Kₐ, between 2 and 10, preferably between 2 and 5, for the disinfection and the cleansing of an acute or chronic skin wound.

The weak sulphonic acid has also proven particularly useful in removing bacterial biofilm and necrotic and/or infected tissues present in skin lesions, preferably in acute or chronic skin ulcers. The experiments performed by the inventors demonstrated that the weak sulphonic acid is able to penetrate the bacterial biofilm with an exceptional surfactant effect towards its components, allowing for its complete removal without giving thermic or painful sensations.

Therefore, the particularly mild action of the weak sulphonic acid allows to obtain excellent results of disinfection and removal of the biofilm and necrotic and/or infected tissues without causing side effects, such as the burning sensation. Consequently, the weak sulphonic acid can be applied autonomously by the patient, without the intervention of specialised staff, such as doctors and nurses, thus overcoming the limitations of known compositions.

The invention also concerns a composition comprising the sulphonic acid in mixture with at least a non-aqueous proton acceptor, preferably a solvent and/or a substance/solvent with basic characteristics, hence capable of accepting protons which allows to adjust, preferably to reduce, the acidity of the composition according to the invention. Preferably, the sulphonic acid is a weak sulphonic acid.

In a preferred embodiment, the least one non-aqueous proton acceptor is selected from: dimethyl sulfoxide, isopropyl alcohol, 3-methoxy-3-methyl-1-butanol, propylene carbonate, anhydrous sodium carbonate, ethylenediaminetetraacetic acid tetrasodium salt, sodium gluconate, anhydrous silicon dioxide, tetraethoxysilane, polyethylene glycol and mixtures thereof.

The composition of the invention comprises the sulphonic acid at a concentration between 50% and 90% w/w, preferably between 60% and 80% w/w.

The composition of the invention comprises the non-aqueous proton acceptor in an amount of 5% to 50% w/w, preferably 10% to 40% w/w.

In some embodiments a minor amount of water is added, up to a maximum of 5% w/w, if needed in case the sulphonic acid is not completely soluble in the non-aqueous proton acceptor or mixture of non-aqueous proton acceptors.

### BRIEF DESCRIPTION OF THE FIGURES

The invention can be better understood and implemented with reference to the attached drawings illustrating an exemplary and non-limiting form of implementation, in which:

FIG. 1 is a photograph of a Petri dish, showing a halo of inhibition of the microbial growth around the deposition zone of a composition containing 4-ethylbenzenesulphonic acid (70%), water (5%) and DMSO (25%) w/w, according to the invention.

FIG. 2 is a photograph of a Petri dish, showing a halo of inhibition of the microbial growth around the deposition zone of a composition containing 4-methylbenzenesulphonic acid (70%), water (5%) and DMSO (25%) w/w, according to the invention.

FIG. 3 is a photograph of a Petri dish, showing a halo of inhibition of the microbial growth around the deposition zone of a composition according to the invention comprising 70% 4-dodecylbenzenesulphonic acid and 2 propanol (30%).

An analogue results is obtained with a composition comprising 70% 4-dodecylbenzenesulphonic acid and DMSO (30%).

FIG. 4 is a photograph showing the effect of treating a chronic wound present on a big toe with a composition according to the invention.

FIG. 5 is a photograph showing the effect of treating a chronic wound present on the leg of a patient with a composition according to the invention.

### DETAILED DESCRIPTION OF THE INVENTION

The invention concerns compositions containing a sulphonic acid having the general formula (A):

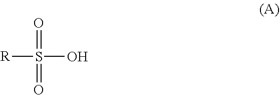

wherein R is an arene group, preferably a benzene group, wherein the arene group or benzene group is optionally substituted with one or more substituents independently selected from alkyl, OH and alkoxy, such as methoxy, wherein the substituent is preferably alkyl having a number of carbon atoms from 1 to 20 and the alkyl chain is at position 3 or 4 of the benzene ring, for use in removing a bacterial biofilm and/or necrotic or infected tissue from acute or chronic, preferably chronic, skin lesions or from oral cavities.

The invention concerns compositions containing a sulphonic acid having the general formula (A):

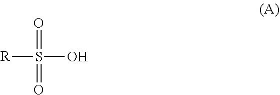

wherein R is an arene group, preferably a benzene group, wherein the arene group or benzene group is optionally substituted with one or more substituents independently selected from alkyl, OH and alkoxy, such as methoxy, wherein the substituent is preferably alkyl having a number of carbon atoms from 1 to 20 and the alkyl chain is at position 3 or 4 of the benzene ring, for use in disinfecting acute or chronic skin lesions.

Preferably, the composition further comprises a non-aqueous proton acceptor.

Preferably, the non-aqueous proton acceptor is at least one component, selected from: dimethyl sulfoxide, isopropyl alcohol, 3-methoxy-3-methyl-1-butanol, propylene carbonate, anhydrous sodium carbonate, ethylenediaminetetraacetic acid tetrasodium salt, sodium gluconate, anhydrous silicon dioxide, tetraethoxysilane, polyethylene glycol and mixtures thereof.

Preferably, the non-aqueous proton acceptor is a liquid at room temperature. In a preferred embodiment, the non-aqueous proton acceptor is a solvent selected from: dimethyl sulfoxide, isopropyl alcohol, 3-methoxy-3-methyl-1-butanol, propylene carbonate, polyethylene glycol and mixtures thereof.

More preferably, the non-aqueous proton acceptor is a solvent for the sulphonic acid of the composition.

Most preferably, the non-aqueous proton acceptor is or comprises dimethyl sulfoxide.

In particular, the composition contains at most 5 wt. % water, preferably is substantially free from water.

In embodiments, the sulphonic acid has an acid dissociation constant (Kₐ) between 2 and 700, preferably between 2 and 200, more preferably between 2 and 100, even more preferably between 2 and 10, most preferably between 2 and 5.

In embodiments, the sulphonic acid is selected from benzenesulphonic acid, guaiacolsulphonic acid, 2-phenolsulphonic acid, 3-phenolsulphonic acid, 4-phenolsulphonic acid and alkylbenzenesulphonic acid having general formula (I).

In embodiments, the composition further comprises another sulphonic acid according to general formula (A), wherein R is an arene group, preferably a benzene group, wherein the benzene group is optionally substituted with one or more substituents independently selected from alkyl, OH and alkoxy, such as methoxy, wherein the substituent is preferably alkyl having a number of carbon atoms from 1 to 20 and the alkyl chain is at position 3 or 4 of the benzene ring, wherein the sulphonic acid and the other sulphonic acid are different. The composition contains a mixture of the sulphonic acid according to general formula (A) and another sulphonic acid according to general formula (A).

In embodiments, the amount of sulphonic acid or the total amount of sulphonic acids is 50% to 90% w/w, preferably 60% to 80% w/w, based on the total weight of the composition.

In embodiments, the non-aqueous proton acceptor component is in an amount of 5% to 50% w/w, preferably 10% to 40% w/w, based on the total weight of the composition.

Preferably, the composition contains substantially no sulphuric acid.

In embodiments, the molar ratio of sulphonic acid to the salt thereof is at least 10:1, more preferably 20:1, most preferably 50:1, in particular the salt thereof is substantially not present. The salt of the sulphonic acid may be any salt, including sodium, potassium, ammonium, etc..

In embodiments, the use comprises applying the composition onto a bacterial biofilm and/or necrotic or infected tissue of the skin lesion.

In embodiments, wherein the use comprises removing the composition from the skin lesion within a time between 1 second-30 minutes after applying the composition.

In particular embodiments, the use comprises removing the composition from the skin lesion within a time between 1 second-10 minutes, preferably within 5 minutes, more preferably within 1 minute, after applying the composition. This use is preferred in case the sulphonic acid has a Kₐ in the range 2-100, in particular in case the sulphonic acid has a Kₐ in the range 10-100.

By selecting the non-aqueous proton acceptor and adjusting the relative amounts of sulphonic acid and non-aqueous proton acceptor the application time window can be adjusted.

In particular embodiments, the use comprises removing the composition from the skin lesion within a time between 1 second-30 minutes, preferably between 1 minute-30 minutes, more preferably between 5 minutes-30 minutes, after applying the composition. This use is preferred in case the sulphonic acid has a Kₐ lower than 100, in particular a Kₐ lower than 10. By selecting the non-aqueous proton acceptor and adjusting the relative amounts of sulphonic acid and non-aqueous proton acceptor the application time window can be adjusted.

In a particular embodiment, the invention concerns an alkylbenzenesulphonic acid having general formula (I)

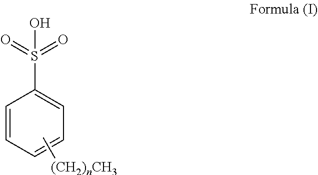

for use in removing a bacterial biofilm and/or necrotic or infected tissues from acute or chronic, preferably chronic, skin lesions.

In formula (I) n is an integer from 0 to 20, preferably from 1 to 20, preferably from 2 to 13. Preferably, n equals 0, 1, 2, 3, 4, 5, 6, 7, 8, 9, 10, 11, 12, 13, 14, 15, 16, 17, 18, 19 or 20. The alkyl chain can be at position 3 or 4 of the benzene ring.

Preferably, the alkylbenzenesulphonic acid of formula (I) has an acid dissociation constant Kₐ between 2 and 10, preferably between 2 and 5.

In a preferred embodiment, the alkyl chain has n=0, 1 or 11.

In a preferred embodiment, the alkyl chain is at position 4 of the benzene ring.

In a preferred embodiment, the alkyl chain has n from 0 to 20, preferably from 1 to 13, and is positioned at position 4 of the benzene ring.

In one embodiment of the invention, n equals 0, 1, or 11 and the alkyl chain is at position 3 or 4 of the benzene ring, preferably at position 4. In a particularly preferred embodiment, the alkylbenzenesulphonic acid of formula (I) is 4-ethyl-benzene-sulphonic acid.

In a preferred embodiment, the alkylbenzenesulphonic acid is 4-dodecyl-benzene-sulphonic acid, which among organic sulphonic acids is one of the weakest:

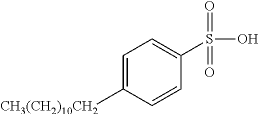

having an acid dissociation constant, Kₐ, estimated in the range 2-3. This means that about 57% of the acid form remains undissociated upon contact with the biofilm (values estimated by solving the second order equation X²+Kₐ×X−70×Kₐ=0 where X represents the proton concentration (H+) concentration). The neutral form therefore has the ability to easily penetrate the non-polar parts and in particular the lipid components of the biofilm. At the same time, the dissociation occurring in presence of water produces a dehydrating effect thanks to the high hydration energy of the proton, able to sequester water molecules present in the biofilm and in the microbial species it contains.

The effectiveness of action of dodecylbenzenesulphonic acid against the biofilm most likely results from the delicate and unpredictable balance between these two actions.

This effect was observed with particular reference to 4-dodecyl-benzene-sulphonic acid but is transferrable to all weak alkylbenzenesulphonic acids, preferably with an acid dissociation constant between 2 and 100.

The alkylbenzenesulphonic acid of formula (I) therefore exerts a disinfectant action on acute and/or chronic skin lesions (wounds and/or ulcers), providing a removal of the biofilm and necrotic and/or infected tissues but without causing side effects, such as the burning sensation. Consequently, the weak sulphonic acid can be applied autonomously by the patient, without the intervention of specialized staff, such as doctors and nurses, thus overcoming the limits of known compositions.

This action is due to the surfactant capacities of the acid that make it able to penetrate the biofilm present on the lesion allowing for the detachment of necrotic tissues and the complete removal through simple washing with water. Furthermore, the surfactant action together with the effect of acid dissociation contributes to the elimination of microorganisms, in particular bacteria and fungi, present in the biofilm.

Other sulphonic acids, for example containing at position 4 non-polar groups such as 3-methylbenzenesulphonic acid or 4-ethylbenzenesulphonic acid, with values of the acid dissociation constant in the order of 600, also have considerable antimicrobial activity even if they have a lower penetrating effect and lower surfactant activity than 4-dodecylbenzenesulphonic acid due to the reasons listed above and to the longer alkyl chain.

Tests performed on patients' biofilm indeed revealed a good antimicrobial activity even if lower than that of 4-dodecylbenzenesulphonic acid.

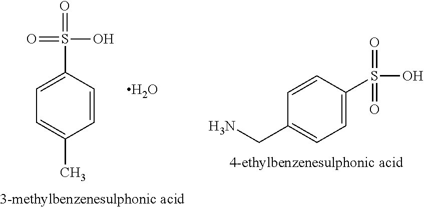

In another aspect, the invention concerns a composition comprising a sulphonic acid of formula (A), preferably an alkylbenzenesulphonic acid of formula (I), as active principle, preferably in combination with suitable non-aqueous co-formulants, preferably co-formulants acting as non-aqueous proton acceptors and able to adjust, in particular to reduce, the acidity of the composition.

The composition according to the invention is able to remove the biofilm and necrotic tissues from infected areas of the skin within few tens of seconds from the application. The composition can be applied directly to the area to be treated by any mean suitable to allow its distribution.

After a contact time of a few minutes, the composition can be readily removed from the skin or from the mucosa by using simple gauze and washing the surface of treated skin or mucosa with a physiological solution or with a stream of sterile water.

Therefore, by applying the composition according to the invention to a skin lesion it is possible to obtain the same effects obtainable through one surgical debridement, without however the well-known drawbacks associated with this latter procedure. This new and effective therapeutic possibility is usable, with significant benefit also directly by the patient, in cases of mild, medium and high severity.

The composition according to the invention, comprising the sulphonic acid of formula (A), preferably the alkylbenzenesulphonic acid of formula (I), can be formulated as a solution or as a gel and can be easily applied to acute and chronic skin ulcers.

A surprisingly unexpected property exhibited by the composition according to the invention is that this one is able to act on the biofilm and on necrotic or infected tissues, causing their disintegration and allowing for their easy and painless removal with a simple gauze and a stream of water or saline solution after a few minutes from application and thus avoiding complicated, painful and expensive surgical procedures.

The action of the composition is evidently due to the penetrating and surfactant power of the alkylbenzenesulphonic acid towards the components of the biofilm and it is also due to the subsequent release of hydrogen ions (H⁺) or protons which, having a high enthalpy of hydration (−1130 KJ/mole), cause dehydration of the microbial species that make up the biofilm or proliferate in infected tissues. This mechanism of action occurs regardless of the microbial species present and makes the composition according to the invention active against any microbial species, be it bacterial, fungal or viral.

Furthermore, the inventors have foreseen the use of the sulphonic acid of formula (A), in particular the alkylbenzenesulphonic acid of formula (I), in combination with small quantities of solvents or non-aqueous proton acceptors which allow to adjust, and more exactly to reduce, the acidity of the composition according to the invention. By lowering the concentration of protons released by the sulphonic acid, the latter can be efficiently employed in the also autonomous treatment of acute and chronic skin lesions and ulcers in which the biofilm is present, to yield the desired disruptive effect while eliminating the infective microbial species, without producing any damage to the healthy surrounding tissues.

In one embodiment, the composition comprises sulphonic acid, preferably alkylbenzenesulphonic acid, in a quantity between 50% and 90% w/w, preferably between 60% and 80% w/w.

Preferably, the composition further comprises a solvent acting as non-aqueous proton acceptor selected from: dimethyl sulfoxide, isopropyl alcohol, 3-methoxy-3-methyl-1-butanol, and propylene carbonate, present in the composition in a quantity between 10% and 50% (w/w), preferably between 20% and 40% w/w. Optionally, a minor amount of water is added as a solvent for the sulphonic acid.

In one embodiment, the composition comprises a mixture of solvents selected among the ones listed above. In this, the concentration from 10% to 50% or 20% to 40% will be divided among the solvents used.

In one embodiment, the composition is formulated as a gel. In in this case, amorphous silica is added to the diluted solutions of sulphonic acid, in an amount between 1 and 10% (w/w) to obtain the product in gel form.

In a further embodiment of the composition according to the invention—the rheology of the gel is optimized by addition of tetraethoxysilane, which acts as a cross-linking agent.

The preparation procedure of the different formulations is not described in details below, as the chemical components of the various formulations can be added to sulphonic acid following a variable order and yet without altering the properties of the final solution.

By way of exemplary, but non-limiting, example of the invention below are described two in vitro antimicrobial activity tests (Example 1) and the procedures for the treatment of skin lesions based on the use of the composition according to the invention (Example 2).

### Formulations

Exemplary Formulations of the invention are as follows:
Composition 1: Benzenesulphonic acid (70%), water (5%) and DMSO (25%) w/w
Composition 2: Phenolsulphonic acid (70%), water (5%) and DMSO (25%) w/w
Composition 3: Guaiacolsulphonic acid (70%), water (5%) and DMSO (25%) w/w
Composition 4: 4-ethylbenzenesulphonic acid (70%), water (5%) and DMSO (25%) w/w
Composition 5: 4-methylbenzenesulphonic acid (70%), water (5%) and DMSO (25%) w/w
Composition 6: 70% 4-dodecylbenzenesulphonic acid and 2-propanol (30%) w/w

pKₐ and Kₐ values of sulphonic acids are given in the Table 1 below:

TABLE 1
acid dissociation constant of sulphonic acids

| Sulphonic Acid | pKₐ | Kₐ |
|---|---|---|
| Benzene sulphonic acid | −6.5 | 3160 |
| Phenol sulphonic acid (o, m, p) | <−3 | >1000 |
| 4-methylbenzene sulphonic acid | −2.8 | 630 |
| 4-Ethylbenzenesulphonic acid | −2.8 | 630 |
| 4-dodecylbenzenesulphonic acid | −0.45 ± 0.50 | 3 |

The pKₐ and the Kₐ values are based on literature references, which include:
E. P. Serjeant and B. Dempsey (eds.), Ionization Constants of Organic Acids in Solution, IUPAC Chemical Data Series No. 23, Pergamon Press, Oxford, UK, 1979;
Part of Journal of Research of the National Bureau of Standards. Volume 31, December 1943, pH VALUES OF ACID.SALT MIXTURES OF SOME AROMATIC SULFONIC ACIDS AT VARIOUS TEMPERATURES AND A CRITERION OF COMPLETENESS OF DISSOCIATION, By Walter j. Hamer. Gladys D. Pinching, and S. F. Acree;
Guthrie, J. P. Hydrolysis of esters of oxy acids: pKₐ values for strong acids. Can. J. Chem. 1978, 56, 2342-2354;

### Example 1—In Vitro Antimicrobial Activity Test

The antimicrobial activity of the formulations of the composition according to the invention was tested against the following strains of microorganisms (purchased from Diagnostic International Distribution S.p.A.): Pseudomonas aeruginosa ATCC 15442, Staphylococcus aureus ATCC 6538, Escherichia coli ATCC 10536, Enterococcus hirae ATCC 10541, Candida albicans ATCC 10231. Mixtures of the different strains of microorganisms were prepared, having concentrations expressed in colonies forming units (CFU) ranging from 1.5×10¹²-5.5×10¹² for each species. 100 μl samples of the mixture were seeded in Petri dishes containing TSA (Tryptone Soya Agar) solid culture medium. Sowing was carried out according to a known and standardized analytical method, that is depositing the liquid sample on the surface of the agar through a micropipette and distributing the liquid sample on the surface of the agar using sterile glass beads. Subsequently, 50 μl aliquots of the six formulations of the composition according to the invention were deposited in a central area of the agar of each Petri dish. The plates were then incubated at 37° C. for 24 h.

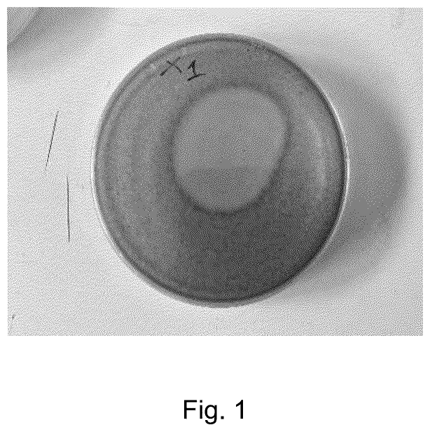

FIG. 1 shows a Petri dish marked X1, where a 50 μl aliquot of the formulation containing 4-ethylbenzenesulphonic acid (70%), water (5%) and DMSO (25%) w/w was deposited.

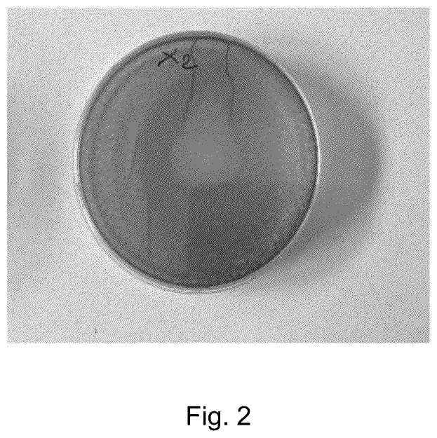

FIG. 2 shows a Petri dish marked X2, where a 50 μl aliquot of the formulation containing 4-methylbenzenesulphonic acid (70%), water (5%) and DMSO (25%) w/w was deposited.

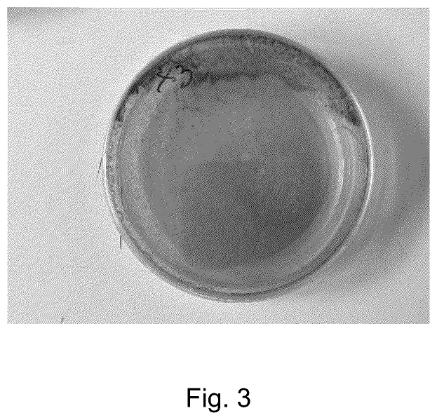

FIG. 3 shows a Petri dish marked X3, where a 50 μl aliquot of the formulation containing 70% 4-dodecylbenzenesulphonic acid and 2-propanol (30%) was deposited.

Analogous results were obtained with the formulation containing 70% 4-dodecylbenzenesulphonic acid and DMSO (30%)

After incubation, the plates were examined in order to evaluate the microbial proliferation (colony formation) and the width of the halo of inhibition (i.e., the size of the portion of land in which microbial proliferation was inhibited) surrounding the agar zone on which the samples of the six formulations were deposited. As shown in the Figures, evident inhibition zones of the microbial growth, surrounding the deposit zones of the formulations according the invention, were observed in all of the Petri dishes. With a greater halo for composition 6. The analytical result obtained indicates that the different formulations are able to inhibit the growth of 10¹¹ CFU of gram-positive bacteria, gram-negative bacteria and of the fungal species Candida albicans.

### Example 2—Treatment of Patients Affected by Chronic Skin Ulcers

The composition according to the invention was tested on over 20 volunteer patients, by applying a treatment protocol comprising the following steps:
a) Preparing the wound by rinsing the wound and drying it prior to applying the formulation;
b) Applying the formulation;
c) Waiting for about 5 to 10 minutes;
d) Removal of formulation from wound with a simple gauze and a stream of water or saline solution.

Tests performed on compositions 4 and 5 gave worse results, despite the proven antimicrobial activity verified in vitro. The difference between in vitro and in vivo tests is related to the presence of the biofilm against which the composition 6 subject of the invention shows a better activity.

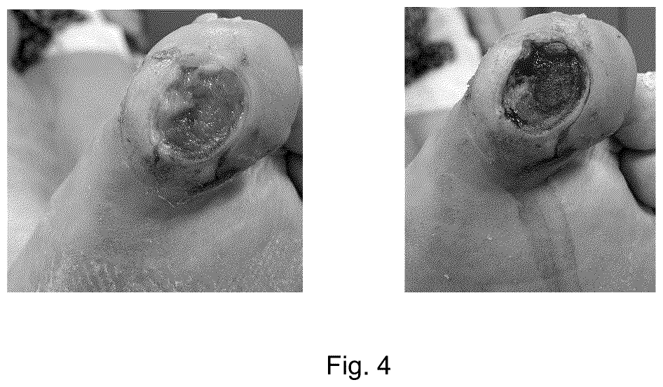

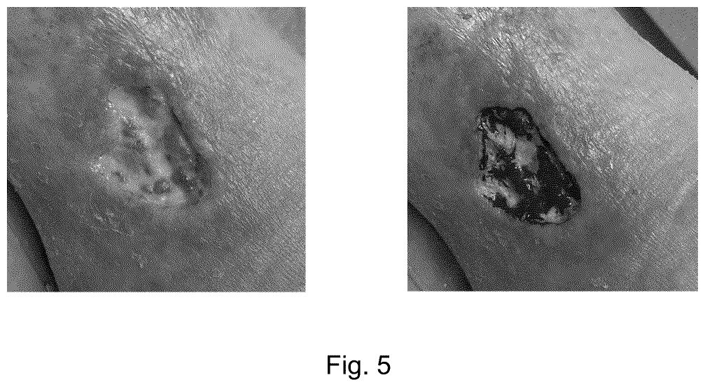

Examples of the effectiveness on the patients of composition 6 containing 4-dodecylbenzenesulphonic acid and 30% isopropyl alcohol are reported in FIGS. 4 and 5.

FIG. 4. The left image shows a chronic ulcer of the radius of the left foot in a diabetic patient. The bottom of the ulcer is covered entirely of yellowish, soft slough, more thick in the left portion. On the right, the same portion of the foot after treatment with composition 6 subject of the invention (containing 70% 4-dodecylbenzenesulphonic acid and 2-propanol (30%)). The bottom of the ulcer appears completely cleansed as the slough has been removed.

FIG. 5. The left image shows a chronic medial malleolar ulcer of venous origin. The bottom is almost entirely covered with hard yellowish slough. Right image shows, after treatment with composition 6 subject of the invention (containing 70% 4-dodecylbenzenesulphonic acid and 2-propanol (30%)), the bottom of the ulcer, which appears cleansed and grainy as the slough has been removed.

Granulation of the bottom of the lesion was achieved in all treated patients.

Even further improved results are obtained with the following formulations 7 and 8 comprising 4-dodecylbenzenesulphonic acid and 2-propanol or Dimethyl sulfoxide (DMSO):

Formulation 7

| Component | Percentage by weight |
|---|---|
| 4-dodecylbenzenesulphonic acid | 70-90% |
| 2-propanol | 10-30% |
| SiO₂ (0.3 microns in average diameter) | 1-3% |
| TEOS | 0.5-1% |

Formulation 8

| Component | Percentage by weight |
|---|---|
| 4-dodecylbenzenesulphonic acid | 70-90% |
| Dimethyl sulfoxide | 10-30% |
| SiO₂ (0.3 microns in average diameter) | 1-3% |
| TEOS | 0.5-1% |

The composition includes amorphous silica SiO₂ and tetraethoxylane (TEOS) and is formulated as a gel. The gel formed composition enhances the local treatment of the wound as the composition remains stationary on the wound during the application.

Therefore, the application of the composition 6 according to the invention containing 4-dodecylbenzenesulphonic acid diluted with the appropriate solvents provided in all cases a complete restoration of the tissues in the lesion, promoting its healing. The treatment protocol described above can potentially be applied to all patients, thus avoiding complicated, expensive and potentially risky surgical procedures. Furthermore, the treatment with the composition according to the invention can reduce the need for antibiotic therapies, which are substantially expensive and associated with the increasingly emerging phenomenon of antibiotic resistance.

1. A composition comprising a sulphonic acid having the general formula (A): 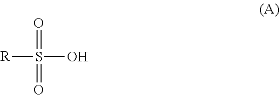
wherein R is an arene group, preferably a benzene group, wherein the arene group or benzene group is optionally substituted with one or more substituents independently selected from alkyl, OH and alkoxy, such as methoxy, wherein the substituent is preferably alkyl having a number of carbon atoms from 1 to 20 and the alkyl chain is at position 3 or 4 of the benzene ring, for use in removing a bacterial biofilm and/or necrotic or infected tissue from acute or chronic, preferably chronic, skin lesions or from oral cavities.

2. A composition comprising a sulphonic acid having the general formula (A): 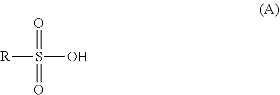
wherein R is an arene group, preferably a benzene group, wherein the arene group or benzene group is optionally substituted with one or more substituents independently selected from alkyl, OH and alkoxy, such as methoxy, wherein the substituent is preferably alkyl having a number of carbon atoms from 1 to 20 and the alkyl chain is at position 3 or 4 of the benzene ring, for use in disinfecting acute or chronic skin lesions.

3. The composition for use according to claim 1 or claim 2, wherein the composition further comprises a non-aqueous proton acceptor.

4. The composition for use according to any one of the preceding claims, wherein the non-aqueous proton acceptor is at least one component, selected from: dimethyl sulfoxide, isopropyl alcohol, 3-methoxy-3-methyl-1-butanol, propylene carbonate, anhydrous sodium carbonate, ethylenediaminetetraacetic acid tetrasodium salt, sodium gluconate, anhydrous silicon dioxide, tetraethoxysilane, polyethylene glycol and mixtures thereof.

5. The composition for use according to any one of the preceding claims, wherein the non-aqueous proton acceptor is or comprises dimethyl sulfoxide.

6. The composition for use according to any one of the preceding claims, wherein the composition contains at most 5 wt. % water, preferably is substantially free from water.

7. The composition for use according to any one of the preceding claims, wherein the sulphonic acid has an acid dissociation constant (Kₐ) between 2 and 700, preferably between 2 and 200, more preferably between 2 and 100, even more preferably between 2 and 10, most preferably between 2 and 5.

8. The composition for use according to any one of the preceding claims, wherein the composition further comprises another sulphonic acid according to general formula (A), wherein R is an arene group, preferably a benzene group, wherein the benzene group is optionally substituted with one or more substituents independently selected from alkyl, OH and alkoxy, such as methoxy, wherein the substituent is preferably alkyl having a number of carbon atoms from 1 to 20 and the alkyl chain is at position 3 or 4 of the benzene ring, wherein the sulphonic acid and the other sulphonic acid are different.

9. The composition for use according to any one of the preceding claims, wherein the amount of sulphonic acid or the total amount of sulphonic acids is 50% to 90% w/w, preferably 60% to 80% w/w, based on the total weight of the composition.

10. The composition for use according to any one of the preceding claims, wherein the non-aqueous proton acceptor component is in an amount of 5% to 50% w/w, preferably 10% to 40% w/w, based on the total weight of the composition.

11. The composition for use according to any one of the preceding claims, wherein the composition contains substantially no sulphuric acid.

12. The composition for use according to any one of the preceding claims, wherein the molar ratio of sulphonic acid to the salt thereof is at least 10:1, more preferably 20:1, most preferably 50:1, in particular the salt thereof is substantially not present.

13. The composition for use according to any one of the preceding claims, wherein the sulphonic acid is an alkylbenzenesulphonic acid having general formula (I) 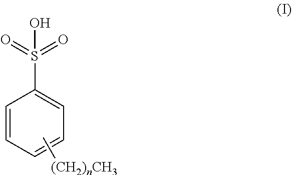
wherein n is an integer from 0 to 20, preferably from 1 to 20, more preferably from 2 to 13, and the alkyl chain is at position 3 or 4 of the benzene ring, for use in removing a bacterial biofilm and/or necrotic or infected tissue from acute or chronic, preferably chronic, skin lesions.

14. The composition for use according to claim 13, wherein n equals 0, 1 or 11.

15. The composition for use according to any one of the preceding claims 13-14, wherein the alkyl chain is at position 4 of the benzene ring.

16. The composition for use according to any one of the preceding claim 13, wherein n is from 0 to 20, preferably from 1 to 13, and the alkyl chain is positioned at position 4 of the benzene ring.

17. The composition for use according to claim 13, wherein n is equal to 0, 1, 2 or 12 and the alkyl chain is in position 3 or 4 of the benzene ring, preferably in position 4.

18. The composition for use according to any one of the preceding claims 13-17, the alkylbenzenesulfonic acid having an acid dissociation constant between 2 and 5.

19. The composition for use according to claim 13, the sulphonic acid being selected from: 4-dodecyl-benzene-sulfonic acid, methyl-benzene-sulfonic acid and ethyl-benzene-sulfonic acid.

20. The composition for use according to any one of the preceding claims, wherein the use comprises applying the composition onto a bacterial biofilm and/or necrotic or infected tissue of the skin lesion.

21. The composition for use according to any one of the preceding claims, wherein the use comprises removing the composition from the skin lesion within a time between 1 second-30 minutes after applying the composition.

22. The composition for use according to any one of the preceding claims, for disinfecting acute or chronic skin lesions.

23. Composition for use according to any one of the preceding claims, further comprising amorphous silica and, preferably, tetraethoxylane, and being formulated as a gel.

24. A composition comprising a sulphonic acid having the general formula (A): 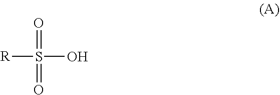
wherein R is an arene group, preferably a benzene group, wherein the benzene group is optionally substituted with one or more substituents independently selected from alkyl, OH and alkoxy, such as methoxy, wherein the substituent is preferably alkyl having a number of carbon atoms from 1 to 20 and the alkyl chain is at position 3 or 4 of the benzene ring, in an amount of 50% to 90% w/w, preferably 60% to 80% w/w; and at least one proton acceptor selected from: dimethyl sulfoxide, isopropyl alcohol, 3-methoxy-3-methyl-1-butanol, propylene carbonate, anhydrous sodium carbonate, ethylenediaminetetraacetic acid tetrasodium salt, sodium gluconate, polyethylene glycol, anhydrous silicon dioxide, tetraethoxysilane, and mixtures thereof; the non-aqueous proton acceptor in an amount of 5% to 50% w/w, preferably 10% to 40% w/w, wherein preferably the composition further comprising amorphous silica and, more preferably, tetraethoxylane, and being formulated as a gel.

25. The composition according to claim 24, wherein the non-aqueous proton acceptor is or comprises dimethyl sulfoxide.

26. The composition according to any one of the preceding claims 24-25, wherein the composition contains at most 5 wt. % water, preferably is substantially free from water.

27. The composition according to any one of the preceding claims 24-26, wherein the sulphonic acid has an acid dissociation constant (Kₐ) between 2 and 700, preferably between 2 and 200, more preferably between 2 and 100, even more preferably between 2 and 10, most preferably between 2 and 5.

28. The composition according to any one of the preceding claims 24-27, wherein the composition further comprises another sulphonic acid according to general formula (A), wherein R is an arene group, preferably a benzene group, wherein the benzene group is optionally substituted with one or more substituents independently selected from alkyl, OH and alkoxy, such as methoxy, wherein the substituent is preferably alkyl having a number of carbon atoms from 1 to 20 and the alkyl chain is at position 3 or 4 of the benzene ring, wherein the sulphonic acid and the other sulphonic acid are different.

29. The composition according to any one of the preceding claims 24-28, wherein the composition contains substantially no sulphuric acid.

30. The composition according to any one of the preceding claims 24-29, wherein the molar ratio of sulphonic acid to the salt thereof is at least 10:1, more preferably 20:1, most preferably 50:1, in particular the salt thereof is substantially not present.

31. The composition according to any one of the preceding claims 24-30, wherein the sulphonic acid is an alkylbenzenesulphonic acid having general formula (I) 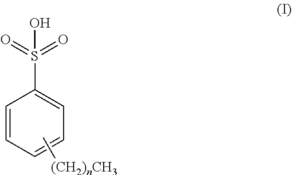
wherein n is an integer from 0 to 20, preferably from 1 to 20, more preferably from 2 to 13, and the alkyl chain is at position 3 or 4 of the benzene ring, for use in removing a bacterial biofilm and/or necrotic or infected tissue from acute or chronic, preferably chronic, skin lesions.

32. The composition according to claim 31, wherein n equals 0, 1 or 11.

33. The composition according to claim 31 or 32, wherein the alkyl chain is at position 4 of the benzene ring.

34. The composition according to claim 31, wherein n is from 0 to 20, preferably from 1 to 13, and the alkyl chain is positioned at position 4 of the benzene ring.

35. The composition according to claim 31, wherein n is equal to 0, 1, 2 or 12 and the alkyl chain is in position 3 or 4 of the benzene ring, preferably in position 4.

36. The composition according to any one of the preceding claims 31-35, the alkylbenzenesulfonic acid having an acid dissociation constant between 2 and 5.

37. The composition according to claim 31, the sulphonic acid being selected from: 4-dodecyl-benzene-sulfonic acid, methyl-benzene-sulfonic acid and ethyl-benzene-sulfonic acid.

38. The composition according to any one of the preceding claims 31-37, for disinfecting acute or chronic skin lesions.

39. Composition according to any one of the preceding claims, further comprising amorphous silica and, preferably, tetracthoxylane, and being formulated as a gel.
